(19) United States
(12) Patent Application Publication
(10) Pub. No.: US 2026/0199206 A1
(43) Pub. Date: Jul. 16, 2026

(54) IMPROVED STRAIGHTENING PROCESS FOR KERATIN FIBERS

(71) Applicant: L'OREAL, Paris (FR)

(72) Inventor: Ayano TAKEDA, Kawasaki-shi (JP)

(21) Appl. No.: 19/135,700

(22) PCT Filed: Nov. 17, 2023
(86) PCT No.: PCT/JP2023/042279

§ 371 Date: Jun. 4, 2025

(30) Foreign Application Priority Data
Dec. 9, 2022 (JP) 2022-197247
Jan. 18, 2023 (FR) 2300474

(51) Int. Cl. A61K 8/365; A45D 7/04; A61K 8/42; A61K 8/44; A61Q 5/06

(52) U.S. Cl. CPC A61K 8/365; A45D 7/04; A61K 8/42; A61K 8/44; A61Q 5/06; A45D 2200/25

(57) ABSTRACT

The present invention relates to a process for straightening keratin fibers such as hair, comprising the steps of: (i) applying a composition onto the keratin fibers; (ii) optionally rinsing off the keratin fibers with or without drying the keratin fibers after being rinsed off; and (iii) straightening the keratin fibers at a temperature of more than 50° C., preferably more than 100° C., and more preferably more than 150° C., wherein the composition comprises (a) at least one compound selected from the group consisting of (a) at least one compound selected from the group consisting of glyoxylic acid, glyoxylic acid solvates, glyoxylic acid salts, glyoxylic acid esters, glyoxylic acid amides, and mixtures thereof; (b) at least one betaine compound; and (c) water. The present invention can improve a process for straightening keratin fibers such as hair based on heating the keratin fibers with glyoxylic acid or a derivative thereof, by providing longer-lasting straightening effects.

### TECHNICAL FIELD

The present invention relates to a process, in particular a cosmetic process, for straightening keratin fibers such as hair.

### BACKGROUND ART

Straightening keratin fibers such as hair by heating the keratin fibers with an iron is popular for making unruly keratin fibers manageable and making keratin fibers have less volume and look more desirable. Such a straightening method using a heating iron is convenient for quick styling, but the style lasts only short period of time, e.g., until the next shampooing. A long-lasting straightening method is required for many consumers.

On the other hand, conventional permanent straightening methods using a reducing agent and an alkaline agent, as well as an oxidizing agent, with heating, can provide keratin fibers with a long-lasting style, but they have several drawbacks such as a long processing time, damage to the keratin fibers, and malodor.

WO 2014/131469 discloses a process for straightening keratin fibers by heating with an iron, without using reducing and oxidizing agents, wherein glyoxylic acid is used to straighten the keratin fibers.

### DISCLOSURE OF INVENTION

There is still a need to improve the straightening of keratin fibers based on heating the keratin fibers with glyoxylic acid or a derivative thereof.

An objective of the present invention is to provide an improved process for straightening keratin fibers such as hair, based on heating the keratin fibers with glyoxylic acid or a derivative thereof, which can provide the keratin fibers with longer-lasting straightening effects.

The above objective of the present invention can be achieved by a process for straightening keratin fibers such as hair, comprising the steps of:
(i) applying a composition onto the keratin fibers;
(ii) optionally rinsing off the keratin fibers, with or without drying the keratin fibers after being rinsed off; and
(iii) straightening the keratin fibers at a temperature of more than 50° C., preferably more than 100° C., and more preferably more than 150° C.,

wherein

the composition comprises
(a) at least one compound selected from the group consisting of glyoxylic acid, glyoxylic acid solvates, glyoxylic acid salts, glyoxylic acid esters, glyoxylic acid amides, and mixtures thereof;
(b) at least one betaine compound; and
(c) water.

It is preferable that the above composition be applied onto wet keratin fibers.

The pH of the above composition may be 7 or less, preferably 6 or less, and more preferably 5 or less.

The amount of the (a) compound(s) in the above composition may be from 0.1% to 50% by weight, preferably from 0.5% to 40% by weight, and more preferably from 1% to 30% by weight, relative to the total weight of the composition.

The (b) betaine compound may be selected from the group consisting of trimethylglycine, carnitine, and L-proline betaine or stachydrine, and mixtures thereof.

The amount of the (b) betaine compound(s) in the above composition may be from 0.01% to 15% by weight, preferably from 0.05% to 10% by weight, and more preferably from 0.1% to 5% by weight, relative to the total weight of the composition.

The amount of the (c) water in the above composition may be from 20% to 99% by weight, preferably from 30% to 95% by weight, and more preferably from 40% to 90% by weight, relative to the total weight of the composition.

The above composition may further comprise (d) at least one compound, different from the (a) compound, selected from the group consisting of keto-acids, salts thereof, and mixtures thereof.

The (d) compound may be selected from levulinic acid, a salt thereof, and a mixture thereof.

The amount of the (d) compound in the above composition may be from 0.1% to 15% by weight, preferably from 0.5% to 10% by weight, and more preferably from 1% to 5% by weight, relative to the total weight of the composition.

The above composition may not comprise any ammonia or a thiol compound, or may comprise less than 1% by weight, preferably less than 0.5% by weight, and more preferably less than 0.1% by weight of ammonia or a thiol compound, relative to the total weight of the composition.

The above composition may not comprise any reducing agent or oxidizing agent, or may comprise less than 1% by weight, preferably less than 0.5% by weight, and more preferably less than 0.1% by weight of a reducing agent or an oxidizing agent, relative to the total weight of the composition.

The present invention also relates to a composition for straightening keratin fibers, such as hair, with heating, comprising:
(a) at least one compound selected from the group consisting of glyoxylic acid, glyoxylic acid solvates, glyoxylic acid salts, glyoxylic acid esters, glyoxylic acid amides, and mixtures thereof;
(b) at least one betaine compound; and
(c) water.

The present invention also relates to a kit for straightening keratin fibers such as hair, comprising:

at least one heater, preferably a heating iron, which is capable of providing the keratin fibers with a temperature of more than 50° C., preferably more than 100° C., and more preferably more than 150° C.; and

at least one composition comprising
(a) at least one compound selected from the group consisting of glyoxylic acid, glyoxylic acid solvates, glyoxylic acid salts, glyoxylic acid esters, glyoxylic acid amides, and mixtures thereof;
(b) at least one betaine compound; and
(c) water.

The present invention also relates to a use of (b) at least one betaine compound in a composition for straightening keratin fibers such as hair, with heating, comprising:
(a) at least one compound selected from the group consisting of glyoxylic acid, glyoxylic acid solvates, glyoxylic acid salts, glyoxylic acid esters, glyoxylic acid amides, and mixtures thereof; and
(c) water

in order to lengthen the straightening effects for keratin fibers by the composition, in particular in order to further maintain the straight shape of the keratin fibers or to prevent or reduce waving of the keratin fibers.

### BEST MODE FOR CARRYING OUT THE INVENTION

After diligent research, the inventors have found that it is possible to provide an improved process for straightening keratin fibers such as hair, based on heating the keratin fibers with glyoxylic acid or a derivative thereof, which can provide the keratin fibers with longer-lasting straightening effects.

Thus, the present invention mainly relates to a process for straightening keratin fibers such as hair, comprising the steps of:
(i) applying a composition onto the keratin fibers;
(ii) optionally rinsing off the keratin fibers with or without drying the keratin fibers after being rinsed off; and
(iii) straightening the keratin fibers at a temperature of more than 50° C., preferably more than 100° C., and more preferably more than 150° C.,

wherein

the composition comprises
(a) at least one compound selected from the group consisting of glyoxylic acid, glyoxylic acid solvates, glyoxylic acid salts, glyoxylic acid esters, glyoxylic acid amides, and mixtures thereof;
(b) at least one betaine compound; and
(c) water.

The present invention can improve a process for straightening keratin fibers such as hair, based on heating the keratin fibers with glyoxylic acid or a derivative thereof, by providing longer-lasting straightening effects.

The present invention can provide keratin fibers such as hair with longer-lasting straightening effects. Thus, the present invention can lengthen or prolong the straightening effects for keratin fibers provided by using a composition including only glyoxylic acid or a derivative thereof. In particular, the present invention can further maintain the straight shape of keratin fibers, or can prevent or reduce loss of the straight shape of keratin fibers over time.

For example, if keratin fibers are originally wavy, the present invention can provide better prevention for the keratin fibers returning to the original wavy shape, after straightening the keratin fibers, in particular after shampooing.

In the present invention, the use of at least one betaine compound can lengthen or prolong the straightening effects for the keratin fibers provided by glyoxylic acid or a derivative thereof. In particular, the use of at least one betaine compound can further maintain the straight shape of the keratin fibers provided by glyoxylic acid or a derivative thereof, or can prevent or reduce waving of the keratin fibers.

The present invention can also provide keratin fibers with sufficient immediate straightening effects. Thus, the keratin fibers treated with the present invention can have less volume or less frizz due to the reduction of curls of the keratin fibers. The straightening effects provided by the present invention can last a long time, for example, even after shampooing the keratin fibers. Thus, the present invention can also provide the shape of keratin fibers with good resistance to shampooing.

The present invention can provide keratin fibers with less damage as compared to conventional straightening processes, because the present invention does not need to use any reducing/oxidizing agents. Therefore, it can be easy to comb the keratin fibers treated by the present invention. Thus, the keratin fibers treated with the present invention can be easy to manage. Also, the keratin fibers treated by the present invention can be stronger than those treated by conventional straightening processes.

In addition, the present invention can use no or very little ammonia or a thiol compound, and therefore, odor during the use of the present invention can be reduced as compared to the conventional processes which require the use of ammonia or a thiol compound. Also, the present invention can have good usability, for example, short processing time.

Hereafter, the present invention will be described in a detailed manner.

### [Process]

The present invention relates to a process for straightening keratin fibers, preferably hair, comprising the steps of:
(i) applying a composition onto the keratin fibers;
(ii) optionally rinsing off the keratin fibers with or without drying the keratin fibers after being rinsed off; and
(iii) straightening the keratin fibers at a temperature of more than 50° C., preferably more than 100° C., and more preferably more than 150° C.,

wherein

the composition comprises
(a) at least one compound selected from the group consisting of glyoxylic acid, glyoxylic acid solvates, glyoxylic acid salts, glyoxylic acid esters, glyoxylic acid amides, and mixtures thereof;
(b) at least one betaine compound; and
(c) water.

The details of the composition used in the process according to the present invention will be explained in the section titled [Composition] below.

The process according to the present invention is for the purpose of straightening keratin fibers such as hair, preferably straightening for a long period of time, and more preferably straightening even after shampooing several times. In this sense, the process according to the present invention can be a semi-permanent straightening process.

It is preferable that the keratin fibers be cleaned before the step (i). This cleaning step may be performed by, for example, shampooing the keratin fibers, followed by rinsing off the keratin fibers. After rinsing off, the keratin fibers may be dried. It is preferable that the keratin fibers are wet just before the step (i).

In the step (i), the composition, which will be described later, is applied to keratin fibers such as hair. The application of the composition may be performed by any means, such as a brush and a comb.

The bath ratio of the applied composition to the keratin fibers may range from 0.1 to 10, more particularly from 0.5 to 5, and preferably between 0.3 and 2. The term “bath ratio” is intended to mean the weight ratio between the total weight of the applied composition and the total weight of the keratin fibers.

It may be possible that, after the application of the composition, the keratin fibers be left as they are for a certain amount of time; typically from 1 minute to 1 hour, preferably from 2 to 30 minutes, and more preferably from 3 to 10 minutes, if necessary, in order to let the composition penetrate into the keratin fibers.

After the step (i), the process according to the present invention may comprise an optional step (ii) of rinsing off the keratin fibers with or without drying the keratin fibers after being rinsed off.

Thus, if the step (ii) is present, the keratin fibers is rinsed off in order to remove the composition from the keratin fibers. The step of rinsing may be performed with water. It is preferable that the keratin fibers be dried after being rinsed off. The drying of the keratin fibers can be performed with a conventional drying means such as a hair dryer.

On the other hand, if the step (ii) is not present, the keratin fibers can be treated by the step (iii) after the step (i) without being rinsed off.

In the step (iii), the keratin fibers, which are preferably dry, are subjected to straightening at a temperature of more than 50° C., preferably more than 100° C., and more preferably more than 150° C., with a heater, preferably a heating iron. This temperature may be less than 250° C., preferably less than 240° C., and more preferably less than 230° C. Thus, this temperature may be more than 50° C. and less than 250° C., preferably more than 100° C. and less than 240° C., and more preferably more than 150° C. and less than 230° C.

As the heating iron, any conventional heating iron can be used. The heating iron can have at least one plate, preferably two plates, which can be heated by, for example, electric heating. The heated plate(s) can be applied onto the keratin fibers and moved along with the direction of the keratin fibers to straighten them.

It is preferable that the heating iron has two plates, and that keratin fibers are sandwiched between the two plates of the heating iron, in which at least one of the plates can be heated, and then the two plates are moved along with the direction of the keratin fibers to straighten them.

It is preferable that the heating iron, in particular a part thereof contacting with keratin fibers, such as a heating plate or heating plates, has a temperature of more than 50° C. and less than 250° C., more preferably more than 100° C. and less than 240° C., and even more preferably more than 150° C. and less than 230° C.

The step (iii) can be performed by one or more strokes of moving the heating iron along with the direction of keratin fibers. The step (iii) can be controlled by not only the temperature of the heating iron but also the number of strokes of the heating iron.

The heating time may depend on the temperature of the heating iron but also on the number of strokes of the heating iron. It may be, for example, from 1 second to 10 minutes, and preferably from a few seconds to 5 minutes.

### [Composition]

The composition to be used for the process according to the present invention, or the composition for straightening keratin fibers, according to the present invention, comprises:
(a) at least one compound selected from the group consisting of glyoxylic acid, glyoxylic acid solvates, glyoxylic acid salts, glyoxylic acid esters, glyoxylic acid amides, and mixtures thereof;
(b) at least one betaine compound; and
(c) water.

It is preferable that the above composition be a cosmetic composition, more preferably a cosmetic composition for straightening keratin fibers, and even more preferably a cosmetic composition for one-step straightening keratin fibers. It is particularly preferable that the keratin fibers be hair.

### (Glyoxylic Acid and Derivatives Thereof)

The composition used for the present invention includes (a) at least one compound selected from the group consisting of glyoxylic acid, glyoxylic acid solvates, glyoxylic acid salts, glyoxylic acid esters, glyoxylic acid amides, and mixtures thereof.

A single type of (a) compound may be used, but two or more different types of (a) compounds may be used in combination.

The (a) compound is selected from the group consisting of glyoxylic acid, glyoxylic acid solvates, glyoxylic acid salts, glyoxylic acid esters, glyoxylic acid amides, and mixtures thereof. In other words, the (a) compound is selected from glyoxylic acid and derivatives thereof.

As glyoxylic acid solvates, mention may be made of, for example, glyoxylic acid hydrates such as glyoxylic acid monohydrate.

As glyoxylic acid salts, mention may be made of, for example, alkaline metal salts or alkaline earth metal salts of glyoxylic acid such as sodium or potassium glyoxylate, and magnesium or calcium glyoxylate.

As glyoxylic acid esters, mention may be made of, for example, alkyl esters of glyoxylic acid such as methyl glyoxylate and ethyl glyoxylate.

As glyoxylic acid amides, mention may be made of, for example, N-glyoxyloylcarbocysteine and N-glyoxyloyl keratin amino acids.

It is preferable to use glyoxylic acid as the (a) compound.

The amount of the (a) compound(s) in the composition used for the present invention may be 0.1% by weight or more, preferably 0.5% by weight or more, and more preferably 1% by weight or more, relative to the total weight of the composition.

On the other hand, the amount of the (a) compound(s) in the composition used for the present invention may be 50% by weight or less, preferably 40% by weight or less, and more preferably 30% by weight or less, relative to the total weight of the composition.

The amount of the (a) compound(s) in the composition used for the present invention may be from 0.1% to 50% by weight, preferably from 0.5% to 40% by weight, and more preferably from 1% to 30% by weight, relative to the total weight of the composition.

### (Betaine Compound)

The composition used for the present invention includes (b) at least one betaine compound. A single type of (b) betaine compound may be used, but two or more different types of (b) betaine compounds may be used in combination.

The term “betaine compound” here means an amphoteric compound having a positively charged cationic moiety and a negatively charged anionic moiety, wherein no hydrogen atom is bound to a positively charged atom in the positively charged cationic moiety and the positively charged cationic moiety may not be adjacent to the negatively charged anionic moiety.

For the purpose of the present invention, the (b) betaine compound here is not a surfactant, which comprises at least one hydrophilic part and at least one hydrophobic part.

The positively charged cationic moiety in the (b) betaine compound includes, but is not limited to, a quaternary ammonium cation, a phosphonium cation, and a sulfonium cation. Preferably, the (b) betaine compound includes a quaternary ammonium cation as the positively charged cationic moiety.

The negatively charged anionic moiety in the (b) betaine compound includes, but is not limited to, a carboxylate anion.

The (b) betaine compound may be selected from the group consisting of trimethylglycine, carnitine, L-proline betaine or stachydrine, and mixtures thereof, and may preferably be trimethylglycine.

The amount of the (b) betaine compound(s) in the composition used for the present invention may be 0.01% by weight or more, preferably 0.05% by weight or more, and more preferably 0.1% by weight or more relative to the total weight of the composition. It may be even more preferable that the amount of the (b) betaine compound(s) in the composition used for the present invention be 0.2% by weight or more relative to the total weight of the composition.

On the other hand, the amount of the (b) betaine compound(s) in the composition used for the present invention may be 15% by weight or less, preferably 10% by weight or less, and more preferably 5% by weight or less relative to the total weight of the composition. It may be even more preferable that the amount of the (b) betaine compound(s) in the composition used for the present invention be 3% by weight or less relative to the total weight of the composition.

The amount of the (b) betaine compound(s) in the composition used for the present invention may range from 0.01% to 15% by weight, preferably from 0.05% to 10% by weight, and more preferably from 0.1% to 5% by weight relative to the total weight of the composition. It may be even more preferable that the amount of the (b) betaine compound(s) in the composition used for the present invention be from 0.2% to 3% by weight relative to the total weight of the composition.

### (Water)

The composition used for the present invention includes (c) water.

Thus, the composition used for the present invention is not anhydrous.

The amount of the (c) water in the composition used for the present invention is 20% by weight or more, preferably 30% by weight or more, and more preferably 40% by weight or more relative to the total weight of the composition.

On the other hand, the amount of the (c) water in the composition used for the present invention may be 99% by weight or less, preferably 95% by weight or less, and more preferably 90% by weight or less relative to the total weight of the composition.

The amount of (c) water in the composition used for the present invention may be from 20% to 99% by weight, preferably from 30% to 95% by weight, and more preferably from 40% to 90% by weight relative to the total weight of the composition.

### (Keto Acid)

The composition for straightening keratin fibers used for the present invention may include (d) at least one compound, different from the (a) compound, selected from keto-acids, salts thereof, and mixtures thereof. Two or more of (d) compounds may be used in combination. Thus, a single type of (d) compound or a combination of different types of (d) compounds may be used.

The keto-acids may be selected from α-keto acids, β-keto acids, γ-keto acids, and mixtures thereof.

The α-keto acids may be compounds represented by the general formula (I):

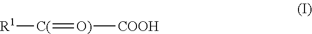

wherein
R¹ denotes a linear or branched C₁-C₆ alkyl group, optionally substituted with an OH, COOH or a halogen atom such as a chlorine or bromine atom.

The examples of α-keto acid include pyruvic acid.

The β-keto acids may be represented by the general formula (II):

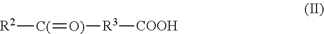

wherein
R² denotes a linear or branched C₁-C₆ alkyl group, optionally substituted with an OH, COOH or a halogen atom such as a chlorine or bromine atom; and
R³ denotes a methylene group, optionally substituted with a halogen atom such as a chlorine or bromine atom.

The examples of β-keto acid include acetoacetic acid.

The γ-keto acids may be represented by the general formula (III):

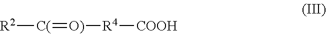

wherein
R² denotes a linear or branched C₁-C₆ alkyl group, optionally substituted with an OH, COOH or a halogen atom such as a chlorine or bromine atom; and
R⁴ denotes an ethylene group, optionally substituted with a halogen atom such as a chlorine or bromine atom.

The examples of γ-keto acids include levulinic acid.

The salts of keto-acid may be alkaline metal salts or alkaline earth metal salts of keto-acid acid such as sodium or potassium salts of keto acid, and magnesium or calcium salts of keto-acid.

It is preferable to use, as the (d) compound(s), γ-keto acid, and more preferably levulinic acid and/or a salt thereof such as sodium levulinate.

The amount of the (d) compound(s) in the composition used for the present invention may be 0.1% by weight or more, preferably 0.5% by weight or more, and more preferably 1% by weight or more, relative to the total weight of the composition.

On the other hand, the amount of the (d) compound(s) in the composition used for the present invention may be 15% by weight or less, preferably 10% by weight or less, and more preferably 5% by weight or less, relative to the total weight of the composition.

The amount of the (d) compound(s) in the composition used for the present invention may be from 0.1% to 15% by weight, preferably from 0.5% to 10% by weight, and more preferably from 1% to 5% by weight, relative to the total weight of the composition.

(pH)

The composition used for the present invention may have a pH of 7 or less, preferably 6 or less, and more preferably 5 or less, which is measured at 25° C.

The composition used for the present invention may have a pH of 3 or more, which is measured at 25° C.

The composition used for the present invention may have a pH of from 3 to 7, preferably from 3 to 6, and more preferably from 3 to 5, which is measured at 25° C.

### (Alkaline Agent)

The composition for straightening keratin fibers, used for the present invention, may comprise at least one alkaline agent. Two or more alkaline agents may be used in combination. Thus, a single type of alkaline agent or a combination of different types of alkaline agents may be used.

The alkaline agent is different from the (b) betaine compound.

It is preferable that the composition used for the present invention include alkaline agent(s), if the (a) compound can function to reduce the pH of the composition.

The alkaline agent may be an inorganic alkaline agent. It is preferable that the alkaline agent be non-volatile. It is preferable that the inorganic alkaline agent be selected from the group consisting of alkaline metal hydroxides; alkaline earth metal hydroxides; and alkaline metal phosphates and monohydrogen phosphates such as sodium phosphate or sodium monohydrogen phosphate.

As examples of the inorganic alkaline metal hydroxides, mention may be made of sodium hydroxide, lithium hydroxide and potassium hydroxide. As examples of the alkaline earth metal hydroxides, mention may be made of calcium hydroxide and magnesium hydroxide. As the inorganic alkaline agent, sodium hydroxide and potassium hydroxide are preferable.

The alkaline agent may be an organic alkaline agent. It is preferable that the organic alkaline agent be selected from the group consisting of monoamines and diamines.

As examples of the monoamines, mention may be made of alkanolamines such as mono-, di- and tri-ethanolamine, comprising 1 to 3 hydroxyalkyl (C₁-C₄) groups. Particularly, alkanolamines may be selected from monoethanolamine, diethanolamine, triethanolamine, monoisopropanolamine, diisopropanolamine, N,N-diméthylethanolamine, 2-amino-2-methyl-1-propanol, triisopropanolamine, 2-amino-2-methyl-1,3-propanediol, 3-amino-1,2-propanediol, 3-dimethylamino-1,2-propanediol, and tris(hydroxymethylamino)methane.

The diamines may be described in the structure (B) below:

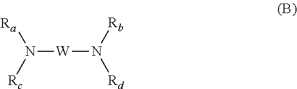

wherein W denotes an alkylene such as propylene optionally substituted by a hydroxyl or a C₁-C₄ alkyl radical, and Ra, Rb, Re and Ra independently denote a hydrogen atom, an alkyl radical or a C₁-C₄ hydroxyalkyl radical, which may be exemplified by 1,3-propanediamine and derivatives thereof.

The amount of the alkaline agent(s) in the composition used for the present invention may be 0.01% by weight or more, preferably 0.05% by weight or more, and more preferably 0.1% by weight or more, relative to the total weight of the composition.

On the other hand, the amount of the alkaline agent(s) in the composition used for the present invention may be 15% by weight or less, preferably 10% by weight or less, and more preferably 5% by weight or less, relative to the total weight of the composition.

The alkaline agent(s) may be used in a total amount of from 0.01% to 15% by weight, preferably from 0.05% to 10% by weight, and more preferably from 0.1% to 5% by weight, relative to the total weight of the composition.

### (Acid)

The composition for straightening keratin fibers, used for the present invention, may comprise at least one acid which is different from the (a) compound or (b) betaine compound. Two or more acids may be used in combination. Thus, a single type of acid or a combination of different types of acids may be used.

The acid may be used to adjust the pH of the composition used for the present invention.

It is preferable that the composition used for the present invention include acid(s), if the (a) compound can function to increase the pH of the composition.

As the acid, mention may be made of any inorganic or organic acids which are commonly used in cosmetic products such as citric acid, lactic acid, sulfuric acid, phosphoric acid or hydrochloric acid (HCl). HCl is preferable.

The amount of the acid(s) in the composition used for the present invention may be 0.01% by weight or more, preferably 0.05% by weight or more, and more preferably 0.1% by weight or more, relative to the total weight of the composition.

On the other hand, the amount of the acid(s) in the composition used for the present invention may be 15% by weight or less, preferably 10% by weight or less, and more preferably 5% by weight or less, relative to the total weight of the composition.

The acid(s) may be used in a total amount of from 0.01% to 15% by weight, preferably from 0.05% to 10% by weight, and more preferably from 0.1% to 5% by weight, relative to the total weight of the composition.

### (Other Optional Ingredients)

The composition used for the present invention may also comprise at least one other optional ingredient, chosen in particular from: oils, preferably polar oils, and more preferably ester oils, in the form of a liquid at 25° C. under atmospheric pressure; solid fatty substances and in particular C₈-C₄₀ esters, C₈-C₄₀ acids, and C₈-C₄₀ alcohols, at 25° C. under atmospheric pressure; thickeners; sunscreens; moisturizers; antidandruff agents; antioxidants; chelating agents; nacreous agents and opacifiers; plasticizers or coalescers; fillers; emulsifiers; polymers, in particular conditioning polymers, such as cationic polymers; fragrances; silanes; crosslinking agents; surfactants including anionic, cationic, amphoteric and nonionic surfactants. The composition can, of course, comprise several cosmetic ingredients appearing in the above list.

The composition used for the present invention may include one or several cosmetically acceptable organic solvents, which may be alcohols: in particular monovalent alcohols such as ethyl alcohol, isopropyl alcohol, benzyl alcohol, and phenylethyl alcohol; diols such as ethylene glycol, propylene glycol and caprylyl glycol; other polyols such as glycerol; and ethers such as ethylene glycol monomethyl, monoethyl, and monobutyl ethers, propylene glycol monomethyl, monoethyl, and monobutyl ethers, and butylene glycol monomethyl, monoethyl, and monobutyl ethers.

Depending on their nature and the purpose of the composition, the above optional ingredient(s) can be present in normal amounts which can be easily determined by those skilled in the art and which can be, for each ingredient, between 0.01% and 80% by weight. Those skilled in the art will take care to choose the ingredients included in the composition, and also the amounts thereof, such that they do not harm the properties of the compositions used for the present invention.

### (Ammonia and Thiol Compound)

It is preferable that the composition used for the present invention be free of ammonia or a thiol compound. The term “free of ammonia or a thiol compound” means that the composition used for the present invention does not include a substantial amount of ammonia or a thiol compound. Preferably the composition used for the present invention includes 1% by weight or less, more preferably 0.5% by weight or less, and even more preferably 0.1% by weight or less of ammonia or a thiol compound, in particular no ammonia or thiol compound.

Due to the very small amount or the absence of ammonia and/or thiol compound, malodor during the use of the composition used for the present invention can be reduced or prevented.

The thiol compound here means a compound which has at least one thiol (—SH) group.

The thiol compound may be a reducing agent. The thiol reducing agent may be chosen from the group consisting of thioglycolic acid and derivatives thereof, in particular esters thereof such as glycerol or glycol monothioglycolate; thiolactic acid and derivatives thereof, in particular esters thereof such as glycerol monothiolactate; 3-mercaptopropionic acid and derivatives thereof, in particular esters thereof such as glycerol 3-mercaptopropionate and ethyleneglycol 3-mercaptopropionate; cysteamine and derivatives thereof, in particular C₁-C₄ acyl derivatives thereof such as N-acetylcysteamine and N-propionylcysteamine; mono-thioglycerol and derivatives thereof, in particular esters; cysteine and derivatives thereof, in particular esters such as N-acetylcysteine, N-alkanoylcysteine and cysteine alkyl esters; thioglycerine and derivatives thereof, in particular s-alkyl derivatives, and salts thereof.

As the above salts, mention may be made of, for example, ammonium salts; primary-, secondary- or tertiary-amine salts; alkaline metal salts; and, alkaline earth metal salts. As the primary-, secondary- or tertiary-amine, for example, monoethanolamine, di-isopropanolamine or triethanolamine, respectively, may be mentioned.

Other examples of the thiol reducing agent include, but are not limited to, sugar N-mercapto alkyl amides such as N-(mercapto-2-ethyl) gluconamide, β-mercaptopropionic acid and derivatives thereof; thiomalic acid; pantheteine; N-(mercaptoalkyl) @-hydroxyalkyl amides such as those described in European Patent Application No. 0 354 835 and N-mono- or N,N-dialkylmercapto 4-butyramides such as those described in European Patent Application No. 0 368 763; aminomercaptoalkyl amides such as those described in European Patent Application No. 0 432 000 and alkylaminomercaptoalkylamides such as those described in European Patent Application No. 0 514 282; (2/3) hydroxy-2 propyl thioglycolate; and the hydroxy-2 methyl-1 ethyl thioglycolate-based mixture (67/33) described in French Patent Application No. 2 679 448.

### (Reducing Agent and Oxidizing Agent)

The composition used for the present invention may comprise a reducing agent; however, it is preferable that the composition used for the present invention comprises a reduced amount of a reducing agent or an oxidizing agent, preferably free of a reducing agent or an oxidizing agent.

The term “free of a reducing agent or an oxidizing agent” means that the composition used for the present invention does not include a substantial amount of a reducing agent or an oxidizing agent. Preferably the composition used for the present invention includes 1% by weight or less, more preferably 0.5% by weight or less, and even more preferably 0.1% by weight or less of a reducing agent or an oxidizing agent, in particular no reducing agent or no oxidizing agent.

The reducing agent may be a thiol reducing agent or a non-thiol reducing agent. The thiol reducing agent is as described above.

The non-thiol reducing agent here means a reducing agent with no thiol group. The non-thiol reducing agent may be chosen from the group consisting of sulfites, bisulfites, sulfinates, phosphines, sugars, reductones and hydrides. The non-thiol reducing agent may be selected from ammonium sulfites and bisulfites as well as metal sulfites and bisulfites, more preferably alkali metal or alkali earth metal sulfites and bisulfites, and more preferably sodium sulfites and bisulfites.

The oxidizing agent may be chosen from hydrogen peroxide, alkali metal bromates, ferricyanides peroxygenated salts, and compounds capable of producing hydrogen peroxide by hydrolysis. For example, the oxidizing agent can be chosen from aqueous hydrogen peroxide solution, urea peroxide, alkali metal bromates and persalts such as perborates and persulphates

### (Form)

The compositions used for the present invention may be in any form suitable for topical application, and in particular in the form of an aqueous, alcoholic or aqueous-alcoholic, or oily solution or suspension; a solution or a dispersion of a lotion or serum type; an emulsion, in particular of liquid or semi-liquid consistency, of O/W, W/O or multiple type (if the composition used for the present invention includes at least one oil); a suspension or emulsion of a soft consistency of cream (O/W) or (W/O) type; an aqueous gel, or any other cosmetic form.

The compositions used for the present invention may be in any galenical forms. For example, the compositions used for the present invention may be in the form of any treatment for hair in general, such as a cream, a lotion, a gel, and the like.

### [Kit]

The present invention also relates to a kit for straightening keratin fibers such as hair, comprising:
at least one heater which is capable of providing the keratin fibers with a temperature of more than 50° C., preferably more than 100° C., and more preferably more than 150° C.; and
at least one composition comprising
(a) at least one compound selected from the group consisting of glyoxylic acid, glyoxylic acid solvates, glyoxylic acid salts, glyoxylic acid esters, glyoxylic acid amides, and mixtures thereof;
(b) at least one betaine compound; and
(c) water.

The heater in the kit is not limited as long as it can heat keratin fibers at a temperature of more than 50° C., preferably more than 100° C., and more preferably more than 150° C. This temperature may be less than 250° C., preferably less than 240° C., and more preferably less than 230° C. Thus, this temperature may be more than 50° C. and less than 250° C., preferably more than 100° C. and less than 240° C., and more preferably more than 150° C. and less than 230° C.

It is preferable that the heater be a heating iron as explained in the above [Process].

The above composition may further comprise (d) at least one compound, different from the (a) compound, selected from the group consisting of keto-acids, salts thereof, and mixtures thereof. The (d) compound may be selected from levulinic acid, a salt thereof, and a mixture thereof.

The above explanations regarding the (a) compound, the (b) betaine compound, and the (c) water, as well as the (d) compound, can apply to those in the above composition used for the kit according to the present invention.

### [Use]

The present invention also relates to a use of (b) at least one betaine compound in a composition for straightening keratin fibers such as hair, with heating, comprising:
(b) at least one compound selected from the group consisting of glyoxylic acid, glyoxylic acid solvates, glyoxylic acid salts, glyoxylic acid esters, glyoxylic acid amides, and mixtures thereof; and
(c) water

in order to lengthen the straightening effects for keratin fibers by the composition, in particular in order to further maintain the straight shape of the keratin fibers or to prevent or reduce waving of the keratin fibers.

The heating is not limited as long as it can heat keratin fibers at a temperature of more than 50° C., preferably more than 100° C., and more preferably more than 150° C. This temperature may be less than 250° C., preferably less than 240° C., and more preferably less than 230° C. Thus, this temperature may be more than 50° C. and less than 250° C., preferably more than 100° C. and less than 240° C., and more preferably more than 150° C. and less than 230° C.

The use of (b) at least one betaine compound can lengthen or prolong the straightening effects for keratin fibers provided by the (a) compound.

The above composition may further comprise (d) at least one compound, different from the (a) compound, selected from the group consisting of keto-acids, salts thereof, and mixtures thereof. The (d) compound may be selected from levulinic acid, a salt thereof, and a mixture thereof.

The above explanations regarding the (a) compound, the (b) betaine compound, and the (c) water, as well as the (d) compound, can apply to those in the above composition used for the use according to the present invention.

### [Additional Invention]

An objective of the present invention can also be realized by a process for straightening keratin fibers such as hair, comprising the steps of:
(i) applying a composition onto the keratin fibers;
(ii) optionally rinsing off the keratin fibers, with or without drying the keratin fibers after being rinsed off; and
(iii) straightening the keratin fibers at a temperature of more than 50° C., preferably more than 100° C., and more preferably more than 150° C.,

wherein

the composition comprises
(a) at least one compound selected from the group consisting of glyoxylic acid, glyoxylic acid solvates, glyoxylic acid salts, glyoxylic acid esters, glyoxylic acid amides, and mixtures thereof in an amount of more than 5% by weight, preferably more than 6% by weight, and more preferably more than 7% by weight relative to the total weight of the composition;
(c) water; and
(d) at least one compound, different from the (a) compound, selected the group water; and consisting of keto-acids (in particular, levulinic acid), salts thereof, and mixtures thereof, in an amount of from 0.1% to 15% by weight, preferably from 0.5% to 10% by weight, and more preferably from 1% to 5% by weight, relative to the total weight of the composition.

The above explanations regarding the (a) compound, the (c) water, and the (d) compound for the composition used for the process according to the present invention can apply to those in the above composition used for the above process.

The above composition does not need to include (b) at least one betaine compound.

The above process can also exert effects that (1) the keratin fibers treated with the above process can provide soft and moisturizing feel, as well as bouncy, (2) the keratin fibers treated with the above process are easily manageable, (3) the above process can prevent keratin fibers from returning to the original shape before the process even after shampooing, and/or (4) the keratin fibers treated with the above process can keep the provided straight shape thereof better even under humid conditions.

### EXAMPLES

The present invention will be described in a more detailed manner by way of examples. However, these examples should not be construed as limiting the scope of the present invention.

### Examples 1-6 and Comparative Examples 1-5

### [Preparations]

Each of the compositions according to Examples 1-6 and Comparative Examples 1-5 was prepared by mixing the ingredients shown in Table 1. The numerical values for the amounts of the ingredients are all based on “% by weight” as raw materials.

TABLE 1

| | Ex. 1 | Ex. 2 | Ex. 3 | Ex. 4 | Ex. 5 | Ex. 6 |
|---|---|---|---|---|---|---|
| Hydroxyethylcellulose | 0.7 | 0.7 | 0.7 | 0.7 | 0.7 | 0.7 |
| Phenoxyethanol | 0.8 | 0.8 | 0.8 | 0.8 | 0.8 | 0.8 |
| Glyoxylic Acid (50 wt %) | 2 | 6 | 6 | 6 | 6 | 10 |
| Trimethylglycine | 0.5 | 0.5 | 1.0 | 1.5 | 2.0 | 0.5 |
| 0.5M HCl aq. | — | — | — | — | — | — |
| Potassium Hydroxide | qs pH 3 | qs pH 3 | qs pH 3 | qs pH 3 | qs pH 3 | qs pH 3 |
| Water | qsp 100 | qsp 100 | qsp 100 | qsp 100 | qsp 100 | qsp 100 |
| Straightening Effects (T0) | 5 | 5 | 5 | 5 | 5 | 5 |
| Area (T0) | 4965 | 5194 | 5225 | 4726 | 5858 | 5302 |
| Straightening Effects (T1sh) | 3 | 4 | 4 | 4 | 3 | 4 |
| Area (T1sh) | 6279 | 5692 | 5575 | 5630 | 5670 | 5696 |

| | Comp. | Comp. | Comp. | Comp. | Comp. |
|---|---|---|---|---|---|
| | Ex. 1 | Ex. 2 | Ex. 3 | Ex. 4 | Ex. 5 |
| Hydroxyethylcellulose | 0.7 | 0.7 | 0.7 | 0.7 | 0.7 |
| Phenoxyethanol | 0.8 | 0.8 | 0.8 | 0.8 | 0.8 |
| Glyoxylic Acid (50 wt %) | — | — | 2 | 6 | 10 |
| Trimethylglycine | — | 0.5 | — | — | — |
| 0.5M HCl aq. | qs pH 3 | qs pH 3 | — | — | — |
| Potassium Hydroxide | — | — | qs pH 3 | qs pH 3 | qs pH 3 |
| Water | qsp 100 | qsp 100 | qsp 100 | qsp 100 | qsp 100 |
| Straightening Effects (T0) | 5 | 5 | 5 | 5 | 5 |
| Area (T0) | 5406 | 5493 | 5156 | 5131 | 5119 |
| Straightening Effects (T1sh) | 1 | 1 | 2 | 2 | 2 |
| Area (T1sh) | 7114 | 7539 | 6896 | 7161 | 7108 |

### [Evaluations]

Each of the above compositions was used to straighten a hair swatch, and then the hair swatch was evaluated, in accordance with the following protocols.
1) A hair watch of Chinese black natural, slightly wavy hair, in a length of 27 cm was shampooed with a SLS Shampoo Composition shown in Table 2 below. The hair swatch was rinsed off thoroughly with tap water.

TABLE 2

| | Wt % |
|---|---|
| Hexylene Glycol | 1 |
| Sodium Chloride | 1 |
| Water | qsp 100 |
| Coco-Betaine | 9 |
| Sodium Laureth Sulfate (70 wt %) | 15 |
| Salicylic Acid | 0.2 |
| Sodium Benzoate | 0.5 |
| Polyquaternium-10 | 0.3 |

2) Each of the compositions according to Examples 1-6 and Comparative Examples 1-5 was applied in an amount of 2 g onto the wet hair swatch.
3) The hair swatch was left for 10 minutes at room temperature (25° C.).
4) The hair swatch was rinsed off thoroughly with tap water.
5) The hair swatch was blow-dried with a hair-dryer (Nobby NB3100, TESCOM DENKI Co., Ltd, Japan) for about 90 seconds until dried.
6) The hair swatch was subjected to straightening by 3 strokes with a hair straightening iron (ADST Premium DS2, Hakko Limited, Japan) at 180° C. for 4 seconds/stroke.
7) The straightening level of the hair swatch was evaluated visually just after step 6 (for straightening effect at TO) under the following criteria.
   5: Very Good
   4: Good
   3: Fair
   2: Poor
   1: Very Poor
   The results are shown in “Straightening Effects (TO)” in Table 1.
8) A photograph of the hair swatch was taken just after step 6, and the area (expressed by the number of pixels) of the hair swatch was determined with an open source, public domain software, Image J. The results are shown in “Area (TO)” in Table 1.
9) The hair swatch was shampooed again with the SLS Shampoo Composition shown in Table 2, rinsed off thoroughly with tap water, and blow-dried with the hair-dryer (for about 90 seconds until dried).
10) The straightening level and area of the hair swatch were evaluated as above again (T1sh). The results are shown in “Straightening Effects (T1sh)” and “Area (T1sh)” in Table 1.

The evaluation results in Table 1 show that the use of a combination of glyoxylic acid and a betaine compound can further maintain the straight shape of hair or reduce the increase in the volume of hair, which reflects de-straightening or waving of hair, than the use of only glyoxylic acid.

In particular, it should be noted that the use of glyoxylic acid can straighten hair, and that the use of a betaine compound in combination with the glyoxylic acid can prevent or reduce the de-straightening or waving of hair. In other words, a betaine compound can contribute to further maintaining the straight shape of hair provided by glyoxylic acid, and can prevent hair from waving or returning to the original shape of the hair, if the original shape thereof is wavy.

### Example 7 and Comparative Example 6

### [Preparations]

Each of the compositions according to Example 7 and Comparative Example 6 was prepared by mixing the ingredients shown in Table 3. The numerical values for the amounts of the ingredients are all based on “% by weight” as raw materials.

TABLE 3

| | | Comp. |
|---|---|---|
| | Ex. 7 | Ex. 6 |
| Isopropyl Myristate | 10 | 10 |
| Glycerin | 4 | 4 |
| Cetearyl Alcohol | 4 | 4 |
| Surfactants | 3.5 | 3.5 |
| Water | qsp 100 | qsp 100 |
| Glyoxylic Acid (50 wt %) | 10 | 10 |
| Levulinic Acid (and) Sodium Levulinate (60 wt %) | 5 | 5 |
| Trimethylglycine | 1 | — |
| Potassium Hydroxide | qs pH 3 | qs pH 3 |
| Ethanol | 3 | 3 |
| Fragrance | 0.2 | 0.2 |
| Straightening Effects (T0) | 5 | 5 |
| Area (T0) | 5453 | 5546 |
| Straightening Effects (T1sh) | 4 | 3 |
| Area (T1sh) | 7657 | 8635 |

### [Evaluations]

The above composition was used to straighten a hair swatch, and then the hair swatch was evaluated, in accordance with the following protocols.
1) A hair watch of Chinese black natural, slightly wavy hair, in a length of 27 cm was shampooed with a SLS Shampoo Composition shown in Table 2 above. The hair swatch was rinsed off thoroughly with tap water.
2) Each of the compositions according to Example 7 and Comparative Example 6 was applied in an amount of 1 g onto the wet hair swatch.
3) The hair swatch was left for 10 minutes at room temperature (25° C.).
4) The hair swatch was rinsed off thoroughly with tap water.
5) The hair swatch was blow-dried with a hair-dryer (Nobby NB3100, TESCOM DENKI Co., Ltd, Japan) for about 90 seconds until dried.
6) The hair swatch was subjected to straightening by 3 strokes with a hair straightening iron (ADST Premium DS2, Hakko Limited, Japan) at 180° C. for 4 seconds/stroke.
7) The straightening level of the hair swatch was evaluated visually just after step 6 (for straightening effect at TO) under the following criteria.
   5: Very Good
   4: Good
   3: Fair
   2: Poor
   1: Very Poor
   The results are shown in “Straightening Effects (TO)” in Table 3.
8) The photograph of the hair swatch was taken just after step 6, and the area (expressed by the number of pixels) of the hair swatch was determined with an open source, public domain software, Image J. The results are shown in “Area (TO)” in Table 3.
9) The hair swatch was shampooed again with the SLS Shampoo Composition shown in Table 2, rinsed off thoroughly with tap water, and blow-dried with the hair-dryer (for about 90 seconds until dried).
10) The straightening level and area of the hair swatch were evaluated as above again (T1sh). The results are shown in “Straightening Effects (T1sh)” and “Area (T1sh)” in Table 3.

The evaluation results in Table 3 also show that a combination of glyoxylic acid and a betaine compound can further maintain straight shaped hair or reduce the increase in the volume of hair, which reflects de-straightening or waving of hair, than the use of only the glyoxylic acid.

Thus, it is clear that the present invention can work not only in a relatively simple aqueous solution (Examples 1-6) but also in an emulsion type formulation (Example 7) which may be close to a commercial hair conditioner.

1-15. (canceled)

16. A method for straightening keratin fibers, comprising:
(i) applying a composition onto the keratin fibers;
(ii) rinsing off the keratin fibers,
(iii) optionally drying the keratin fibers after rinsing; and
(iv) straightening the keratin fibers at a temperature of more than 50° C., wherein the composition comprises:
(a) at least one compound chosen from glyoxylic acid, glyoxylic acid solvates, glyoxylic acid salts, glyoxylic acid esters, glyoxylic acid amides, or mixtures thereof;
(b) at least one betaine compound; and
(c) water.

17. The method of claim 16, wherein the composition is applied onto wet keratin fibers.

18. The method of claim 16, wherein the pH of the composition is 7 or less.

19. The method of claim 16, wherein the amount of the at least one compound (a) chosen from glyoxylic acid, glyoxylic acid solvates, glyoxylic acid salts, glyoxylic acid esters, glyoxylic acid amides, or mixtures thereof the ranges from 0.1% to 50% by weight, relative to the total weight of the composition.

20. The method of claim 16, wherein the amount of the at least one compound (a) chosen from glyoxylic acid, glyoxylic acid solvates, glyoxylic acid salts, glyoxylic acid esters, glyoxylic acid amides, or mixtures thereof the ranges from 1% to 30% by weight, relative to the total weight of the composition.

21. The method of claim 16, wherein the at least one betaine compound (b) is chosen from trimethylglycine, carnitine, L-proline betaine, stachydrine, or mixtures thereof.

22. The method of claim 16, wherein the total amount of the at least one betaine compound (b) ranges from 0.01% to 15% by weight, relative to the total weight of the composition.

23. The method of claim 16, wherein the total amount of the at least one betaine compound (b) ranges from 0.1% to 5% by weight, relative to the total weight of the composition.

24. The method of claim 16, wherein the total amount of water (c) ranges from 20% to 99% by weight, relative to the total weight of the composition.

25. The method of claim 16, wherein the total amount of water (c) ranges from from 40% to 90% by weight, relative to the total weight of the composition.

26. The method of claim 16, wherein the composition further comprises (d) at least one compound, different from the at least one compound (a) chosen from glyoxylic acid, glyoxylic acid solvates, glyoxylic acid salts, glyoxylic acid esters, glyoxylic acid amides, or mixtures thereof, wherein the at least one compound (d) is chosen from keto-acids, salts thereof, or mixtures thereof.

27. The method of claim 26, wherein the at least one compound (d) is chosen from levulinic acid, a salt thereof, or a mixture thereof.

28. The method of claim 26, wherein the total amount of the at least one compound (d) ranges from 0.1% to 15% by weight, relative to the total weight of the composition.

29. The method of claim 26, wherein the total amount of the at least one compound (d) ranges from 1% to 5% by weight, relative to the total weight of the composition.

30. The method of claim 16, wherein the composition does not comprise an ammonia compound or a thiol compound.

31. The method of claim 16, wherein the composition comprises an ammonia compound in a total amount of less than 1% by weight, or a thiol compound in a total amount of less than 1% by weight, relative to the total weight of the composition.

32. The method of claim 16, wherein the composition does not comprise a reducing agent or an oxidizing agent.

33. The method of claim 16, wherein the composition comprises a reducing agent in a total amount of less than 1% by weight, or an oxidizing agent in a total amount of less than 1% by weight, relative to the total weight of the composition.

34. A composition for straightening keratin fibers, comprising:
(a) at least one compound chosen from glyoxylic acid, glyoxylic acid solvates, glyoxylic acid salts, glyoxylic acid esters, glyoxylic acid amides, or mixtures thereof;
(b) at least one betaine compound; and
(c) water.

35. A kit for straightening keratin fibers, comprising:
at least one heater configured to provide heat to the keratin fibers at a temperature of greater than 50° C.; and
at least one composition comprising:
(a) at least one compound chosen from glyoxylic acid, glyoxylic acid solvates, glyoxylic acid salts, glyoxylic acid esters, glyoxylic acid amides, or mixtures thereof;
(b) at least one betaine compound; and
(c) water.
